(19) United States
(12) Patent Application Publication
(10) Pub. No.: US 2009/0043141 A1
(43) Pub. Date: Feb. 12, 2009

(54) Oxidative coupling of methane

(76) Inventors: Terry Mazanec, Solon, OH (US); Wayne Simmons, Plain City, OH (US); John Brophy, Clifton (GB); Fred Pesa, Aurora, OH (US); Anna Lee Y. Tonkovich, Dublin, OH (US); Robert D. Litt, Westerville, OH (US); Dongming Qiu, Dublin, OH (US); Laura J. Silva, Plain City, OH (US); Micheal J. Lamont, Plain City, OH (US); Maddalena Fanelli, Plain City, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 12/156,461

(22) Filed: May 30, 2008

Related U.S. Application Data
(60) Provisional application No. 60/932,260, filed on May 30, 2007.
(60) Provisional application No. 60/962,786, filed on Aug. 1, 2007.
(60) Provisional application No. 60/962,784, filed on Aug. 1, 2007.

(51) Int. Cl. C07C 2/00; B01J 19/00

(52) U.S. Cl. 585/310; 422/198

(57) ABSTRACT

A microchannel apparatus comprising a conduit including a microchannel mixing section, a microchannel reaction section, a microchannel heat transfer section, and a separation section, where the microchannel mixing section includes direct injection inlets, where the microchannel mixing section is downstream from the reaction section, and where the separation section is downstream from the reaction section. Further exemplary embodiments are also disclosed.

### CROSS-REFERENCE TO RELATED APPLICATIONS

This application is a claims priority under 35 U.S.C. §§ 119, 120 to U.S. Provisional Patent Application Ser. No. 60/932,260 filed on May 30, 2007, entitled “OXIDATIVE COUPLING OF METHANE,” and to U.S. Provisional Patent Application Ser. No. 60/962,786 filed on Aug. 1, 2007, entitled “METHODS FOR APPLYING MICROCHANNELS TO SEPARATE METHANE USING LIQUID ABSORBENTS, ESPECIALLY ICONIC LIQUID (IL) ABSORBENTS FROM A MIXTURE COMPRISING METHANE AND NITROGEN,” and further to U.S. Provisional Patent Application Ser. No. 60/962,784 filed on Aug. 1, 2007, entitled “METHODS FOR APPLYING MICROCHANNELS TO SEPARATE GASES USING LIQUID ABSORBENTS, ESPECIALLY ICONIC LIQUID (IL) ABSORBENTS,” the disclosure of each is hereby incorporated by reference.

### GOVERNMENT RIGHTS

Aspects of this invention may have been made with U.S. Government support under Contact DE-FC26-03NT41905 awarded by the United States Department of Energy. The U.S. Government may have certain rights in this invention.

### INTRODUCTION TO THE INVENTION

### Field of the Invention

The present invention is directed to microchannel unit operations and, more specifically, to microchannel unit operations for use in oxidizing methane or methane and other reacting species to form higher molecular weight hydrocarbons and/or oxygenates and removal of those products from effluent streams, while recycling the portion of the effluent stream for further reactions, all conducted within a single microchannel apparatus or within a series of distinct microchannel devices within a single assembly or within a series of distinct microchannel devices that are within separate assemblies.

The invention includes a stage-wise approach for conducting oxidative coupling of methane through two or more stages of reaction, interspersed with at least one separation step. Absorption using ionic fluids is one potential separation useful for this reaction. A similar microchannel apparatus architecture may be used wherein other separation techniques may be used to remove the desired product from an intermediate reaction product stream. For example, a membrane separation may replace absorption with ionic fluids. Alternatively, the desired product may be absorbed onto a solid or reacted to a more stable intermediate to be removed from the apparatus, and then subsequently desorbed or converted back to the desired species.

### BRIEF DESCRIPTION OF THE DRAWINGS

FIG. 1 is a schematic diagram of an exemplary process flow for a microchannel chemical process in accordance with an exemplary embodiment of the instant invention; and

FIG. 2 is a three-dimensional process stream diagram of the microchannel chemical process schematically represented in FIG. 1;

FIG. 3 is an exemplary thermally integrated system for recuperating energy to an absorption- desorption cycle using ionic liquids;

FIG. 4 is an integrated absorption desorption system in a single block to recuperate heat from the two half cycles to reduce the overall parasitic power loss;

FIG. 5 is an alternate system for efficiently transferring energy between absorption and desorption cycle to reduce parasitic power use;

FIG. 6 is an exemplary heat recuperation embodiment in a absorption/desorption process of methane into ionic liquid for separation;

FIG. 7 is an exemplary configuration of microchannels in the heat exchanger core; and

FIG. 8 is an exemplary temperature profile of the superior microchannel heat exchanger.

### DETAILED DESCRIPTION

The exemplary embodiments of the present invention are described and illustrated below to encompass microchannel unit operations for carrying out chemical reactions and separation processes, as well as methods of carrying out chemical reactions and separation processes within an integrated microchannel apparatus. The integrated microchannel apparatus may include integration within an assembly or integration within a plant with distinct assemblies. Of course, it will be apparent to those of ordinary skill in the art that the preferred embodiments discussed below are exemplary in nature and may be reconfigured without departing from the scope and spirit of the present invention. However, for clarity and precision, the exemplary embodiments as discussed below may include optional steps, methods, and features that one of ordinary skill should recognize as not being a requisite to fall within the scope of the present invention.

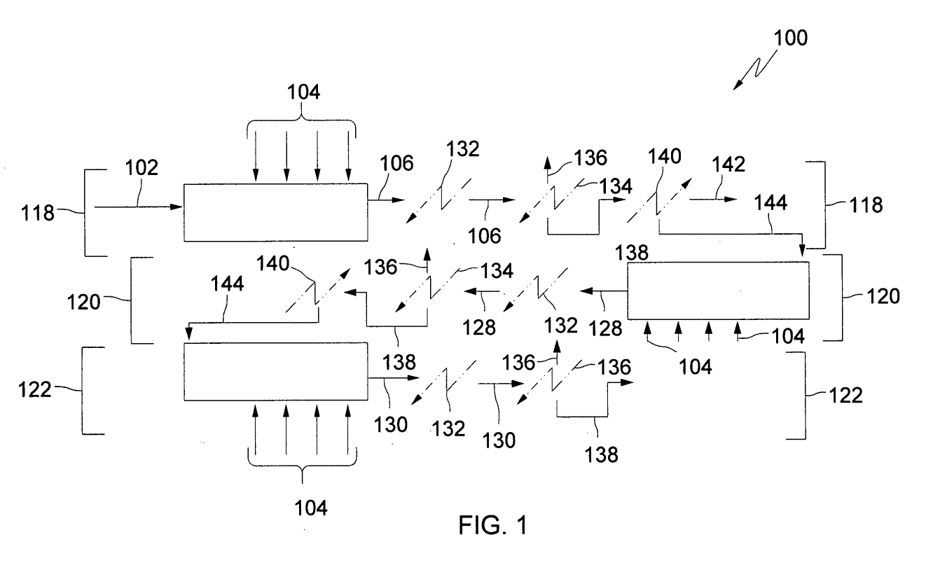

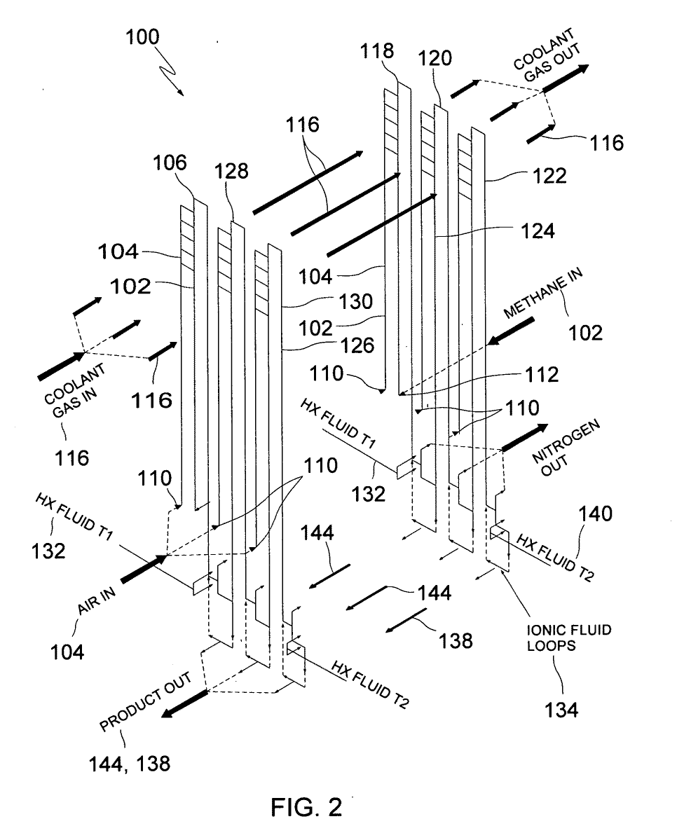

Referencing FIGS. 1 and 2, a first exemplary embodiment of the invention includes a microchannel apparatus 100 carrying out an oxidative coupling of methane reaction. Oxidative coupling of methane may be utilized to convert natural gas streams or other methane-containing streams into higher molecular weight hydrocarbon products. Exemplary hydrocarbon products from oxidative coupling of methane reactions include, without limitation, ethylene, ethane, acetylene, propane, propylene, higher hydrocarbons (such as butane and higher, abbreviated as C₄ and C₄₊ₙ, where n is any integer), acetic acid, and oxygenates. Moreover, this reaction also produces less desired products, such as carbon monoxide and carbon dioxide. Air, oxygen, or another source of oxygen may be used as a reactant. Water, unreacted oxygen, and nitrogen may also be present in the microchannels comprising the reactor portion. In exemplary form, the following is a general reaction equation for the oxidative coupling of methane:

CH₄+O₂→C₂H₆+C₂H₄+C₃₊Hₓ+CO+CO₂+H₂O #1

But Equation #1 is a net equation and is not entirely indicative of the reaction mechanisms behind the production of hydrocarbon products from methane or other light hydrocarbon streams.

The integrated plant technology may also include other reactions, such as oxidative dehydrogenation reactions, oxidation reactions, and others that are particularly advantaged by operating with a highly hydrocarbon rich or lean mixture locally within the reaction section of the system, where the off stoichiometric mixture of reactants produces a selectivity to a desired product higher than that produced with a stoichiometric ratio of feeds. In this embodiment, the excess of either reactant allows to improve the overall reaction selectivity. The integrated plant allows for the efficient recovery of the excess reactant for an overall increase in the yield. The unreacted reactant is captured and recycled to the reaction zone or fed to a series reaction zone to allow for further reaction.

The following equations characterize at least some of the reactions taking place during the overall oxidative coupling of methane reaction.

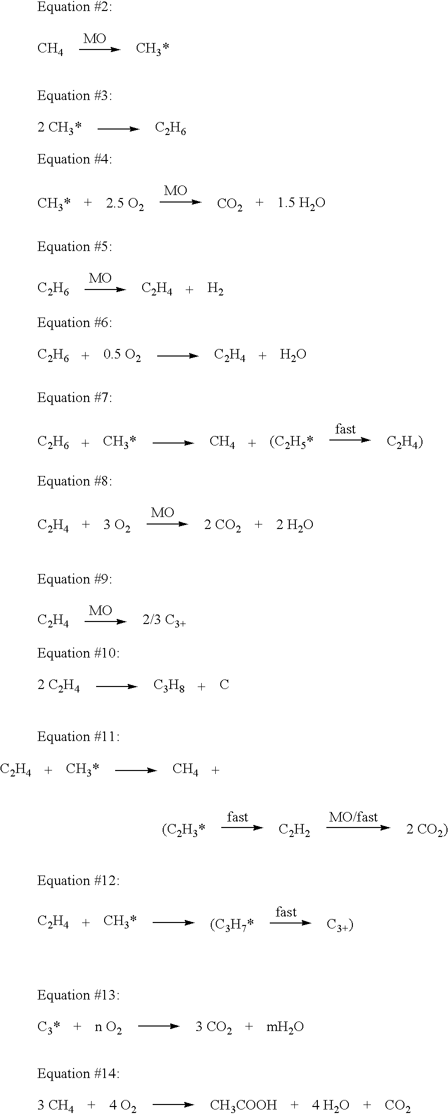

Oxidative coupling of methane reactions, however, suffer from an inverse correlation between selectivity of the desired hydrocarbon product(s) and percent conversion of the methane reactant. Moreover, thermal energy transfer issues, combined with this inverse correlation, have negated profitable commercial implementation of oxidative coupling of methane reactions on a large scale. But the instant invention is operative to overcome these prior barriers.

Other reactions, including oxidation reactions also exhibit a decline in selectivity to the desired product as the conversion of feedstock is increased. These alternate reactions may be operated with a low conversion per pass to improve the overall selectivity to the desired product.

Referring again to FIGS. 1 and 2, the exemplary microchannel apparatus 100 incorporates catalysts (not shown) and operating conditions providing high selectivity toward higher hydrocarbons with acceptable overall conversion of methane. Selectivity in this context refers to the percentage of the desired product, for example, ethane, ethylene, and acetylene in the converted hydrocarbon product stream. Conversion, on the other hand, refers to the percentage of methane that is eventually converted into hydrocarbons. In exemplary form, a methane-containing gaseous stream 102 enters a first set of microchannels 104 that directs the methane-containing stream 102 into intimate communication with a catalyst (not shown) that may be coated along the walls of the microchannels, packed, inserted, or suspended within the microchannels, and/or entrained within the methane-containing stream 102. Exemplary catalysts for use in the instant invention include, without limitation, molybdenum oxides, manganese oxides, vanadium oxides, potassium molybdenum oxides/silicon oxide, copper/gallium ZSM type 5, lithium oxides, and rare earth oxides including samarium oxide, lanthanum oxide, and others in this chemical family. Further exemplary catalysts for use with the instant invention comprise the general formula M₁IM₂Ox, where M₁ is selected from the group comprising Cu, Cr, Fe, Ag, Pd, and M₂ is selected from the group comprising Zn, Zr, Mn, Ce, and rare earth metals. Still further catalysts for use with the instant invention comprise oxides chosen among Al, Si, P, Mg, V, Zr, Ce, Mo Te, Sb, Bi and other transition metals, and mixtures thereof.

An oxygen source for performing the oxidation is mixed with the methane-containing gaseous stream 102. The oxygen source may comprise air, air enriched with oxygen, oxygen, or another source of oxygen. The oxygen source may be mixed with the methane-containing gaseous stream 102 prior to entering the process microchannels or within the process microchannels at one or more locations along the reaction coordinate. A preferred embodiment comprises mixing an oxygen-containing stream 104 with the methane-containing gaseous stream 102 in two or more incremental stages inside of microchannels within which the oxidative coupling of methane reactions are occurring. This mixing may occur before the catalyst is introduced and/or in the presence of the catalyst, resulting in the conversion of some of the methane into various products. The product stream may comprise ethylene, ethane, higher hydrocarbons, carbon dioxide, carbon monoxide, water, and nitrogen (when air is used as the oxygen source).

In an alternate embodiment, the oxygen is supplied through a membrane that either feeds both oxygen and nitrogen or may reduce the amount of nitrogen relative to air. In one embodiment, an oxygen transport membrane is used to feed oxygen into the reaction zone and avoid feeding nitrogen. The nitrogen as separated may be used down stream within the inventive process as a heat transfer fluid, a desorption fluid to remove the separated product from its separation medium, or other uses.

The oxidative coupling of methane net reaction (see Equation #1) is a highly exothermic process and it has been found that process temperatures between 600-1000C. increase the selectivity of the products toward ethylene, ethane and higher hydrocarbons, while reducing the production of undesired byproducts such as carbon oxides. Because temperatures between 600-1000C. have been found to positively impact selectivity, preheating of the methane-containing stream 102 and the oxygen-containing stream 104 is carried out using the product-containing stream 106 in a countercurrent flow relationship. In this exemplary embodiment, oxidative coupling of methane reactions may be carried out at pressures ranging from 0-100 bar, with catalytic contact times ranging from 0.01-400 milliseconds. Exemplary per pass conversions range from 10-50%, where per pass selectivity to ethane +ethylene +acetylene may be greater than 70%.

Referring to FIG. 2, an exemplary three-dimensional flow schematic representative of process flows within the microchannel apparatus 100 shows the repeating nature of these flows. A description of one repeating unit within the apparatus 100 follows. The repeating units are multiplied in order to scale-up the process to the desired production capacity. The lower half of the apparatus 100 includes distributed process channels 110 receiving the oxygen-containing stream 104, while the opposite side of the apparatus includes a process channel 112 receiving the methane-containing stream 102. It should be noted, however, that the streams 102, 104 may enter the apparatus 100 from the same side or from adjacent sides. The feed process channels 102 and 104 are oriented in a countercurrent flow path with respect to the reaction product process channels 106, 128, 130 removing products from the reaction zone. This countercurrent flow orientation is operative to increase the temperature of the reactant streams 110 and 112 along the entire length of the feed process channels by maintaining a ΔT profile between the feed process channels 102, 104 and reaction product process channels 106, 128, 130. However, the reactant streams 102, 104 may be ineffective as a complete heat sink to remove a sufficient amount of the thermal energy of the product stream 106 prior to carrying out the first separation phase. The reactant stream may remove a portion of the energy from the product stream to reduce the product stream temperature and increase the reaction stream temperature Thus, an additional thermal energy transfer stream 116 may be brought into thermal communication with the product stream 106 to remove additional thermal energy from the product stream 106.

A coolant gas stream 116 (i.e., a heat transfer medium) flows cross-current through microchannels that interpose reaction loops 118, 120, 122. In this exemplary embodiment, each reaction loop 118, 120, 122 includes a methane-containing 102 or carbon-containing gaseous stream 124, 126, an oxygen-containing stream 104, and a countercurrent product stream 106, 128, 130. Those skilled in the art will readily understand that liquids may also be utilized in lieu of, or in addition to, gases as a heat transfer medium. Likewise, the flow rates, pressures, and thermal characteristics of the heat transfer medium may be tailored to the particular thermal load, which itself may depend upon the operating conditions of the streams 102, 104, 124, 126 feeding the oxidative coupling of methane reaction. It should be noted, however, that co-current or countercurrent flows of the coolant gas stream 116 (or other heat transfer medium) is also within the scope of the invention, as the microchannels carrying the heat transfer medium may be formed into the microchannel shims in a vertical, as opposed to horizontal, orientation. It should also be noted that the means for heat removal from the exothermic OCM reaction may be an endothermic reaction. One endothermic reaction may be the cracking of ethane to ethylene or the reforming of methane to synthesis gas. The superior heat transfer enabled by microchannel heat exchange between the heat transfer medium and the reactants and products flowing through the reaction loop channels results in excellent thermal control of the reaction that could not be achieved in prior art oxygen coupling of methane processes using conventional equipment. The peak temperature in the reaction portion of the apparatus 100 may be greater than 600C. and may be isothermal or a monotonically increasing function to be controlled to be within 100C. of the average temperature for the reaction portion. The temperature may rise for a portion of the reactor and be nearly isothermal for a portion of the reactor. Nearly isothermal includes reasonable gradients as produced by a real system and are generally considered to be within a 50 C. band. This advanced thermal control of the oxidative coupling of methane reaction or other exothermic reactions enhanced by operating locally in a reaction region well away from a stoichiometric ratio results in enhanced selectivity for hydrocarbon products and reduced yields of carbon oxides. It is generally considered that if a stoichiometric ratio of hydrocarbon to oxygen is 2:1, then 2.5:1 is substantially stoichiometric and a ratio of 3.5:1 or greater is substantially well away from stoichiometric.

Connections to the microchannel apparatus 100 supplying the methane-containing gaseous stream 102, the oxygen-containing streams 104, and the cooling gas streams 116, may utilize manifolds (not shown) to simultaneously distribute/stage/pulse a single input stream across numerous microchannels. For example, a first manifold (not shown) distributes the oxygen-containing stream 104 across the reaction loops 118, 120, 122. This oxygen-source manifold and an inlet coupling (not shown) for the methane-containing stream 102 are mounted to a lower half of the apparatus 100, while the upper half of the apparatus where the highly exothermic reactions are occurring is free of external connections.

Configuring the top half of the apparatus 100 to be free of external connections is a result of thermal design considerations. In general, a vertical temperature profile is established with respect to the apparatus 100, with the highest temperatures being at or near the top of the apparatus 100, while the lower temperatures are at the bottom of the apparatus. The top and bottom may be oriented either up or down with respect to gravity within an assembly. A preferred orientation is to have the hot end point down with respect to gravity and hang from the cold end which is up. Principally, the top half of the apparatus 100 is operated under much greater temperatures in view of the highly exothermic oxidative coupling of methane reactions taking place in this half. By eliminating external connections, such as inlet feeds and outlet piping, the microchannel shims comprising the apparatus 100 are able to expand more evenly and unencumbered at the higher temperature end. The lower half of the apparatus 100, however, is operated at temperatures where thermal expansion considerations are less significant, which is primarily the reason why inlet and outlet connections are found on the lower half. Moreover, the lower half of the apparatus 100 is also where the separation processes are carried out to remove desired product from the product streams 106, 128, 130, vent those by-products not useful for further oxidative coupling of methane reactions, and collect the intermediate products and unreacted methane useful for further oxidative coupling of methane reactions in subsequent reaction loops 120, 122.

Two staged separation processes are carried out in the apparatus 100 in order to remove desired product from the product streams 106 128, 130, vent those by-products not useful for further oxidative coupling of methane reactions, and subsequently react the intermediate products with a source oxygen for further oxidative coupling of methane reactions. The first stage of the separation process includes further cooling the product streams 106, 128, 130 with a heat transfer medium stream 132 prior to and during mixing the product streams with an ionic liquid stream 134.

Ionic liquids are a category of compounds which are made up entirely of organic ions and are liquid at or below process temperatures. Usually, such compounds produce solids with high melting points (commonly known as ‘molten salts’). Ionic liquids differ from ‘molten salts,’ in that they have low melting points, and are liquid at process temperatures. Moreover, they tend to be liquid over a very wide temperature range, with a liquid range of up to about 500C. Ionic liquids are generally non-volatile, with no effective vapor pressure. Most are air and water stable, and are good solvents for a wide variety of inorganic, organic, and polymeric materials. The properties of the ionic liquids can be tailored by varying the cation and anion. Examples of ionic liquids are described, for example, in J. Chem. Tech. Biotechnol., 68:351-356 (1997); Chem. Ind., 68:249-263 (1996); and J. Phys. Condensed Matter, 5: (supp 34B):B99-B106 (1993), Chemical and Engineering News, Mar.30, 1998,32-37; J. Mater. Chem., 8:2627-2636 (1998); and Chem. Rev., 99:2071-2084 (1999), the contents of which are hereby incorporated by reference.

Ionic liquids are used herein to dissolve, suspend, disperse or otherwise immobilize product-complexing metal salts. When a mixture containing the desired products (methane, ethane, ethylene, carbon monoxide, carbon dioxide, etc.) is contacted with such an ionic liquid, the hydrocarbons and carbon containing gases are selectively complexed, forming a metal salt/product complex. Since ionic liquids are non-volatile, the non-complexed products from the oxidative coupling reaction may be easily separated via distillation or other conventional methods. Furthermore, the primary products (ethane, ethylene, etc.) are recovered from the metal salt/product complex via distillation or other conventional methods, allowing the ionic liquid-metal salt solution to be recovered, recycled, and reused.

Referring back to FIGS. 1 and 2, cooling of the products within the product streams 106, 128, 130 is operative to increase the solubility of products within the ionic liquid stream 134. In this first separation stage, the temperature is lowered so that essentially all of the carbon containing species are soluble in the ionic liquid stream 134. In some embodiments, a portion of the carbon containing species are soluble in the ionic liquid and a portion remain insoluble or in equilibrium such that a portion is retained in the gas phase. This may be accomplished by absorbing the hydrocarbon products (such as ethylene, ethane, and benzene) and carbon oxides (carbon monoxide and carbon dioxide) into the ionic liquid stream 134. Heat transfer fluids for use with this first separation process include any heat transfer fluid operative to lower the temperature of the products to a sufficient extent that substantially all or a portion of the carbon containing species within the product stream 106, 128, 130 are dissolved in the ionic liquid stream 134. Exemplary temperature ranges for the ionic liquid stream 134 include ⁻10-200 C., while exemplary temperature ranges for the product streams 106, 128, 130 during the first separation process include ⁻10-100 C. It is understood that solubility of gases within the ionic liquid may follow Henry's law or other gas-liquid equilibrium relationship.

Exemplary techniques for mixing the ionic liquid stream 134 with the product streams 106, 128, 130 include, without limitation, any number of techniques for mixing two fluids in microchannels known in the art, including methods described in U.S. Published Patent Application No. 2005/0133457, which is incorporated herein by reference. As an alternative to mixing, the gas and liquid streams may flow on either side of an adjacent-to-a-porous-contactor for mass transfer of one or more chemical compounds across the porous medium, as exemplified in U.S. Pat. No. 6,126,723, herein incorporated by reference.

A two-phase system is present during the first separation process comprising a gaseous stream 136 and a heavy liquid stream 138. The gaseous stream 136 is predominantly nitrogen, while the heavy liquid stream 138 comprises the ionic liquid as well as dissolved carbon containing species. The gas and liquid phases are separated and directed to different conduits by any number of techniques for phase separation in microchannels known in the art, including methods described in U.S. Pat. Nos. 6,666,909, 6,869,462, 7,051,540, and 6,875,247, which are hereby incorporated by reference. The gaseous stream 136 may be vented from the apparatus 100 or directed to a down-stream apparatus (not shown) where such a stream would be useful. Likewise, the heavy ionic liquid stream 138 is fed into a portion of the apparatus 100 carrying out the second stage of the separation.

The second separation stage includes heating the heavy ionic liquid stream 138 using a second heat transfer fluid stream 140. Heat transfer fluids for use in this second stream 140 include any heat transfer fluid operative to raise the temperature of the heavy ionic liquid stream 138 to a sufficient extent that the vast majority of the hydrocarbon species remain dissolved within a product ionic liquid stream 144, while the other carbon containing species (methane, carbon dioxide, carbon monoxide, etc.) come out of solution to generate a gaseous phase stream 142. In other words, the second separation stage takes a liquid inlet stream 138 having numerous dissolved species and generates a two-phase mixture that is separated into a gaseous stream 142 and a liquid stream 144.

The gaseous stream 142 includes the reactants for the second reaction loop 120 that are brought into contact with an oxygen-containing stream 104 and catalyst (not shown) to carry out further oxidation coupling of methane reactions. The second reaction loop 120 and any other intermediate reaction loop includes a two-stage separation process similar to the foregoing explanation for the first reaction loop 118. The final reaction loop 122, however, includes only a single separation process. In an inventive element of this apparatus the time required to cool the reaction fluid stream to the separation section is preferably less than 250 milliseconds. In some preferred embodiments, the time to cool the fluid is less than 100 milliseconds, in one embodiment the time to cool the reaction fluid stream which contains products after the first reaction stage may be from about 1 to 100 milliseconds.

The absence of successive reaction loops negates the opportunity to carry out a two stage separation process in the final loop 122. Instead, the final loop 122 simply has a single separation process that includes bringing a thermal energy transfer fluid stream 132 into communication with a final products stream 130 that is mixed with an ionic liquid stream 134. As discussed above, mixing of a product stream 130 with an ionic liquid stream 134 results in those carbon containing species dissolving, while the other species remain in gaseous form that are vented from the apparatus 100. The heavy ionic liquid stream 138 is then withdrawn from the apparatus 100 for further separation processes to principally remove the hydrocarbon components, including the desired product. Those remaining carbon containing species within the heavy ionic liquid stream 138 may be recycled (not shown) and redirected into one of more of the reaction loops 118, 120, 122.

Within the apparatus 100, each product ionic liquid stream 142 and the heavy ionic liquid stream 138 from the final loop 122 are directed to an outlet manifold (not shown) that directs the ionic liquid streams to a separation process, or more preferably, a distillation process (not shown) where the hydrocarbon product and other constituents are removed from the ionic fluid. The purified ionic fluid is then returned to the apparatus 100 to comprise the ionic liquid feed stream (not shown).

While the example above has utilized an ionic liquid as the separation medium, other liquid streams or solutions are able to be substituted for the ionic liquid to accomplish the same objective. Metal salt solutions known to selectively bind olefins and acetylenes in the presence of paraffins could be used as the separation medium, for example. Particularly useful solutions are those of Ag, Cu, Au or Ni salts that bind olefins. These solutions can comprise water or other solvent that maintains the proper concentration and oxidation state of the metal ions.

In this exemplary embodiment, the separation zone of the apparatus 100 is less than four meters away from the reaction zone. In an alternate embodiment, the separation zone is conducted in a separate assembly and may be within 200 m of the reaction zone. Moreover, exemplary heat fluxes within the reaction zone (where the reactions are taking place) range from 0.01-30 watts/cm². Further, exemplary dimensions of the apparatus include those dimensions accommodating volumes less than 1.0 m³. However, it is too be understood that scaling provides for apparatuses 100 having volumes greater than 1.0 m³. Further the volume of the apparatus may be greater than 10 m3 and in some embodiments, the volume of the assemblies may be greater than 1000 m3.

An assembly is a system of one or more microchannel devices, which are attached to inlet and outlet macromanifolds for flow of feed and product streams. An assembly may include a pressure vessel which houses one or more microchannel devices. One or more assemblies are integrated into a chemical processing plant through connection with adjacent piping or other equipment. A chemical plant may contain one or more assemblies comprising microchannel devices. Further description of microchannel assemblies and their use are included in US 2005/0175519, US 2005/0249647, and US 2005/0214202, herein incorporated by reference. Many combinations of assemblies may be used for the inventive system. The reaction, heat exchange may be in one assembly, while heat exchange and separation in a second assembly. Regeneration of the separation agent could be in a third assembly or housed in either the first or second assembly. In some embodiments two distinct assemblies are required, in other embodiments three or four distinct assemblies are required. In one embodiment, all unit operations are maintained in the same assembly. There may be more than one device either in series or parallel in the same assembly.

It is also within the scope of the invention to provide a recycle stream that injects small amounts of desired reactants (i.e., higher hydrocarbons) into the methane-containing stream 102 and/or carbon-containing streams 124, 126 to facilitate an autocatalytic reaction.

The apparatus 100 as described above is also useful for reactions other than, or synergistically in combination with, oxidative coupling of methane. For example, the apparatus 100 is amendable to produce hydrocarbons and/or oxygenates from various feed streams such as, without limitation, syngas streams, alkenes, alkynes, oxygenates, and nitrites. In addition, the apparatus 100 can be used for methane conversion to high valued products using multi-functional catalysts/reaction systems by oxidative coupling followed by capture and/or reaction of intermediates (such as ethylene) to remove the intermediates from the reaction environment and reducing their further reaction to undesirable complete oxidation products. This includes, but is not limited to, hydration/oxidation to oxygenates/ethanol, oxidation to acetic acid/esters, and oligomerization to alpha-olefins.

The apparatus 100 may be constructed from a metal or alloys, including Ni-based alloys, suitable for the temperature ranges of interest. The reactor may also be manufactured from a stainless steel and passivated with silica or other dense coating. The reactor may alternatively be manufactured from silicon carbide, glass or quartz. The interior passageways of the apparatus 100 may be coated to create desirable properties of the surfaces in the reactor, heat exchanger, and/or separation passageways. Formation of aluminide coating layers as described in WIPO Publication No. WO/05094983 may be conducted to prevent corrosion of materials of construction for the portion of the apparatus operated at high temperature. For purposes of the instant disclosure, WO/05094983 is incorporated by reference.

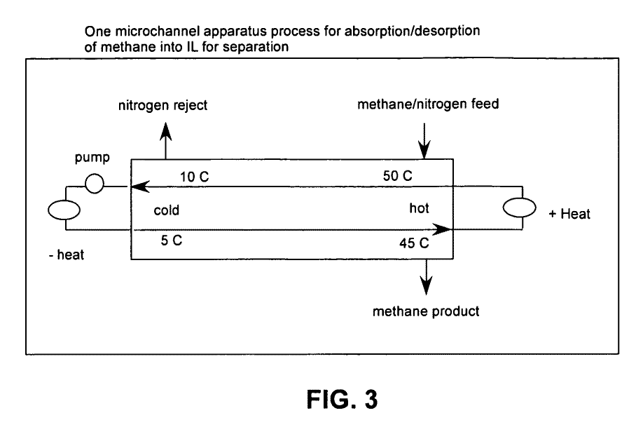

Referring to FIG. 3, an exemplary separation system is shown to minimize power consumption for the purification of methane in a fluid comprising methane and nitrogen. This thermally integrated system is operative to recuperate energy to an absorption-desorption cycle using ionic liquids. As shown in this figure, exemplary approach temperatures are 5 C. at each end of the unit and a larger 40 C. driving force is applied to assist with increasing the capacity difference for methane.

The Henry's law constant for methane at 10 C. of 1480 bar is used along with the minimum Henry's law constant for nitrogen of 20,000 bar. Using an inlet system pressure of 10 bar and a feed gas mixture comprising 80% methane, the liquid mole fraction of methane is 0.016. For a system with a total feed flow rate of 2,000,000 standard cubic feed per day and the 80% feed methane composition, the required flow rate of the ionic liquid, in exemplary form, 1-n-butyl-3-methylimidazolium hexafluorophosphate, is roughly 54,000 L/min at equilibrium to recover roughly 63% of the methane. The reported molecular weight of this ionic liquid is 284 gm/mole. The reported density is roughly 1.37 gm/cc. The volumetric ratio of liquid to gas is roughly 1.4 at the feed inlet. The ratio increases to roughly 7 as the bulk of the methane is absorbed into the ionic liquid. For alternative ionic liquids with more capacity for methane than 1-n-butyl-3-methylimidazolium hexafluorophosphate, the amount of ionic liquid required could be reduced roughly proportionally with the reduction in Henry's law constant.

From the reported maximum Henry's law constant for nitrogen, the minimum purity possible in a single stage is 98.2%. If the actual Henry's law constant is lower than the maximum reported value, then the purity for methane in a single stage may be higher than 98.2%
Purity=(ymethane/Hmethane)/(ymethane/Hmethane+yN2/HN2)
where y is the feed mole fraction and H is the Henry's law constant.
   For a thermal swing process, energy must be added to the gas-fluid mixture to desorb the methane and removed from the fluid to absorb the methane. Using an average heat capacity as reported in the literature of 400 J/mol-K and the reported liquid flow rates then the amount of energy added will be a function of the degree of thermal recuperation.

As shown in Table 1, as the approach temperature is improved at each end of the unit, the total amount of energy required to drive the system is reduced. A preferred process is operated with approach temperatures less than 10 C. to remove the heat, or add the heat to the ionic liquid, or both. A more preferred process will have approach temperatures less than 5C. A more preferred process still may have approach temperatures below 2C. and in some embodiments less than 0.5 C. In one case, the approach temperature at one or both ends may range from 0.05 C. to 0.5 C.

| dT in C. | | | |
|---|---|---|---|
| (approach at | Q-heat | Q-cool | Q total |
| each end) | (MW) | (MW) | (MW) |
| 10 | 17.3 | 17.3 | 34.6 |
| 5 | 8.7 | 8.7 | 17.4 |
| 2 | 3.7 | 3.7 | 7.4 |
| 1 | 1.7 | 1.7 | 3.4 |
| 0.5 | 0.87 | 0.87 | 1.74 |
| 0.1 | 0.17 | 0.17 | 0.35 |

Energy required as a function of approach temperature to capture 63% of a mixture comprising 80% nitrogen and flowing at 2 million standard cubic feet per day using the 1-n-butyl-3-methylimidazolium hexafluorophosphate ionic liquid.

The absorption process requires heat for methane when using 1-n-butyl-3-methylimidazolium hexafluorophosphate as the ionic liquid of roughly 2 kJ/mol of methane. For this separation and the moles of methane absorbed roughly 47 kW of energy will be required during absorption. Using the heat capacity of the ionic liquid, this roughly equates to less than a 0.03 C. temperature loss in the fluid, but this is advantageous as the bulk temperature of this fluid stream is decreasing in the absorption section of the process system. This small increase will result in a slight increase in the log mean temperature difference for the heat exchanger and result in a slightly smaller requirement for surface area which is advantageous. In order to achieve higher methane recovery, more ionic liquid may be required.

TABLE 2
Relationship between methane recovery and flow rate of 1-n-butyl-
3-methylimidazolium hexafluorophosphate ionic liquid absorbent.

| Methane recovery | IL flow rate |
|---|---|
| % | (L/min) |
| 63 | 54550 |
| 70 | 68350 |
| 80 | 95530 |
| 90 | 138300 |
| 95 | 170300 |

TABLE 3
Relationship between methane recovery and flow rate of alternative
ionic liquid absorbent with ten times lower Henry's law constant for
methane absorption than 1-n-butyl-3-methylimidazolium hexafluoro-
phosphate, or namely an H equal to 148 bar.

| Methane recovery | IL flow rate |
|---|---|
| % | (L/min) |
| 63 | 5290 |
| 70 | 6650 |
| 80 | 9340 |
| 90 | 13590 |
| 95 | 16780 |

An alternate exemplary embodiment where the Henry's law constant for methane of 148 bar and a target methane recovery of 90%, then roughly 13,600 L/min of 1-n-butyl-3-methylimidazolium hexafluorophosphate ionic liquid would be required for the 2,000,000 standard cubic feet per day application in this example to recover 90% of the methane with a purity exceeding 98% from a feed mixture of 80% methane. For this case with recuperation of 0.5 C. at each end of the heat exchanger, then less than a 0.5 MW of thermal energy would be required for the methane separation to a purity exceeding 98% from a feed of 80%. For this system the thermal energy content of the purified methane is roughly 17 MW which does not account for losses of conversion from thermal energy to work. Using a typical conversion efficiency of a gas fired power plant of roughly 65%, then the total net work generated from this capacity system is roughly 11 MW. The total parasitic power requirement to drive this system is less than 10% of the total, and for this example less than 5% of the work generated from the purified methane. For the extreme case where a 0.1 C. approach temperature is maintained for the heat exchanger, then with the improved absorbent, likely an ionic liquid, then the total parasitic thermal power required is roughly 0.1 MW which represents on the order of a 1% parasitic power loss. Further it is possible to consider the recovery of a higher fraction of methane beyond 90%. In one embodiment, 92%, or 95%, or 97%, or 99% of the methane is captured from the system. As the absorption capacity increases, the ease of efficiently capturing more of the methane in a one-pass system improves.

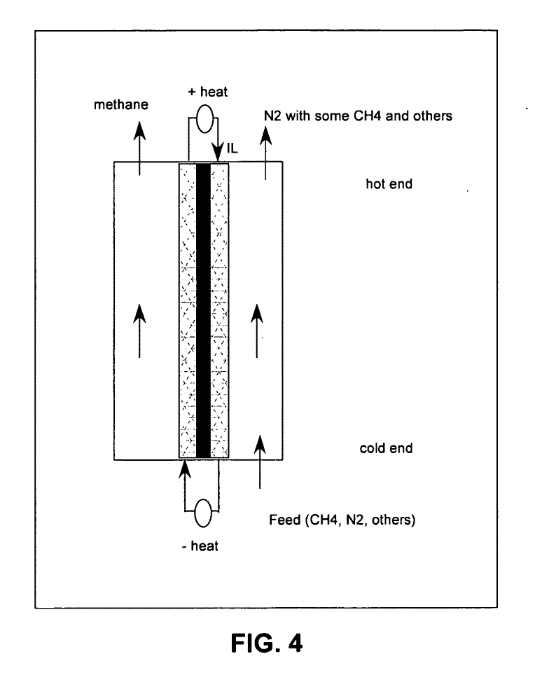

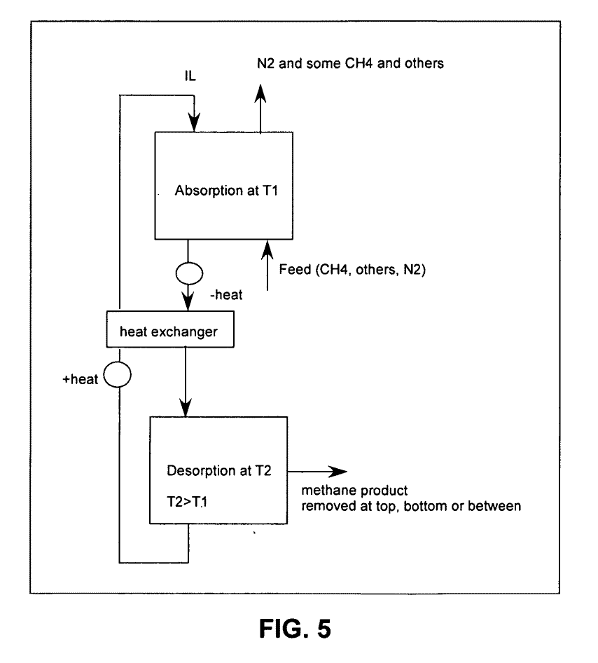

Referring to FIGS. 4 and 5, an alternate exemplary embodiment of a multi-stage absorption system is shown. This system includes a counterflow of an ionic liquid and the feed gas to enable the use of a reduced volume of the ionic liquid absorbent. The heat is recuperated between hot and cold devices or ends of a device is shown in FIG. 4. The recuperation of heat reduces the amount of parasitic energy loss for an advantaged system. The further advantage of the counterflow absorption system is the enablement of multiple stages for separation which reduces the inventory required of the ionic liquid. This approach would require contacting of the two phases in a counterflow mode. The absorber and desorber unit could be separate unit operations as shown in FIG. 5 or integrated in a single unit operation or block as shown in FIG. 4.

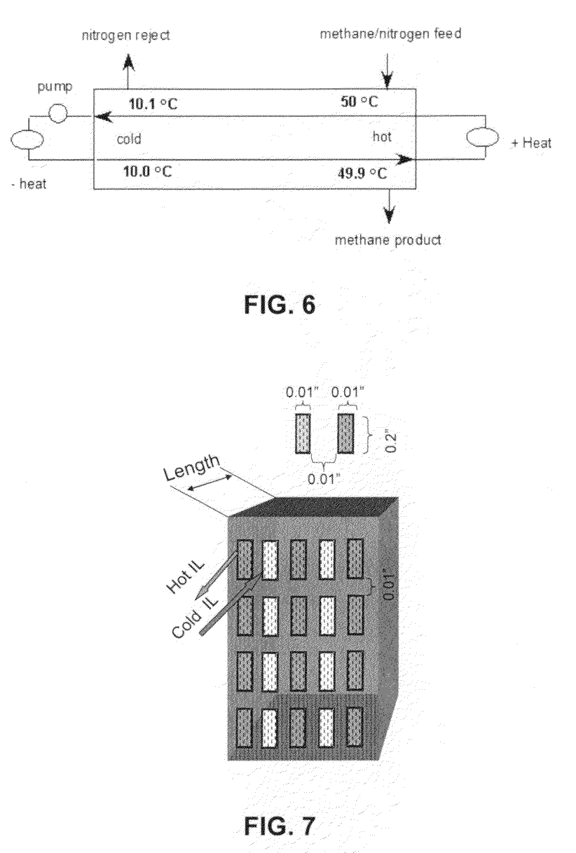

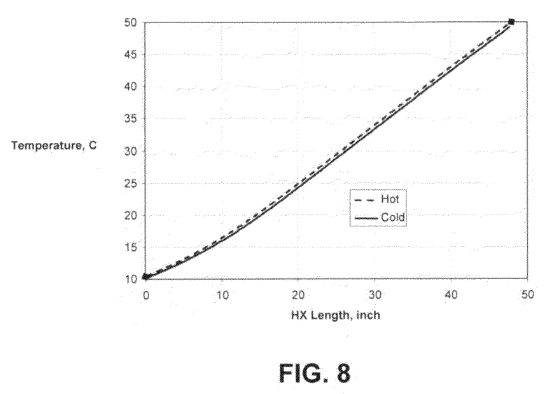

Referencing FIGS. 6 and 7, an exemplary microchannel heat exchanger/recuperater is shown. It is important to reduce the parasitic energy loss in the absorption/desorption process of, for example, methane into ionic liquid for separation in order to make the system operation economical. This means to reduce the energy input “−heat” or “+heat” in the system, which is equivalent to having a heat recuperation with very tight temperature approach at the hot and cold end. Thus, a superior heat exchanger is desirable. The following are exemplary process conditions for operating a microchannel heat exchanger pursuant to this exemplary embodiment:
1. Liquid flow rate: 54,000 L/min; closed loop system
2. CP=1407 J/mol-K
3. density=1.37 gm/cc
4. viscosity=30 cP
5. Tₘₐₓ=50 C.
6. T_{low}=10 C.
7. Thermal conductivity=0.19 W/m-K
8. Approach temperature target 0.1 to 0.25 K on each end.
9. Liquid volume 1000-10000 liter.
10. Material: SS.

As shown in FIGS. 6 and 7, a counter-current flow arrangement is used. The microchannel wall is 0.01″ thick that separates the hot and cold liquids, while the channel gap size is also 0.01″. For the above given flow rate at each side, a total length of 48″ is necessary to achieve a 0.25 K approach temperature at the two ends for a goal of a temperature differential near 40 C. between the hot and cold ends of the absorption and desorption system. For a system requiring a smaller temperature difference to achieve a desired system capacity for the absorbed solute, perhaps 20 C. or 10 C. or more or less difference between maximum and minimum temperature, then the advantaged process may have a shorter heat exchanger length less than 48″ to achieve a very small (<1C.) approach temperature.

TABLE 4
Parameters of the ionic liquid heat exchanger
with 0.25 K approach temperature

| | | |
|---|---|---|
| # of channel | 5000000 | |
| Density | 1370 | kg/m3 |
| Total Flow Rate | 50000 | L/min |
| | 4110000 | kg/h |
| Flow rate/channel | 0.01 | L/min |
| | 822 | g/h |
| Dh | 0.038095238 | in |
| | 0.000967619 | |
| A | 1.29032E−06 | m3 |
| Re | 5.707619881 | |
| Channel volume | 7.86579E−07 | m3 |
| Total liquid volume | 3.933 | m3 |
| Metal volume/channel | 9.439E−07 | m3 |
| (No perimeter) | | |
| | 4.72 | m3 |
| Heat transferred/channel | 12.57665 | Watts |
| Total | 62883250 | Watts |

The building material of the heat exchanger can be any chemically compatible metal or non-metal, as long as its thermal conductivity is in the range 1-500 W/m K, the change in the approach temperature is less than 1 degree Celsius. However, the liquid conductivity has a great effect. For a diluted ionic liquid, for example [bmim][PF6], at a conductivity 0.38 W/m K, the heat exchanger length can be shortened to less than 30″ for the same approach temperature. Thus, for an optimization combining thermal and chemical processes, a diluted ionic liquid with higher thermal conductivity components is an option.

Following from the above description and invention summaries, it should be apparent to those of ordinary skill in the art that, while the methods and apparatuses herein described constitute exemplary embodiments of the present invention, the invention contained herein is not limited to this precise embodiment and that changes may be made to such embodiments without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein. For example, a plurality of reactants refers to two or more of the same reactant.

What is claimed is:

1. A microchannel apparatus comprising:
a conduit including a microchannel mixing section, a microchannel reaction section, a microchannel heat transfer section, and a separation section;
wherein the microchannel mixing section includes direct injection inlets;
wherein the microchannel mixing section is downstream from the reaction section;
wherein the separation section is downstream from the reaction section.

2. The microchannel apparatus of claim 1, wherein the reaction section includes a catalyst supporting an oxidative coupling of methane reaction.

3. The microchannel apparatus of claim 1, wherein the separation section includes a microchannel.

4. The microchannel apparatus of claim 3, wherein the microchannel mixing section, the microchannel reaction section, and the separation section are integrated within a single device.

5. The microchannel apparatus of claim 1, wherein the separation section includes two stages of microchannels, with at least one of the two stages of microchannels supporting separation by phases.

6. The microchannel apparatus of claim 1, wherein the separation section is adapted to carry out at least one of phase separation, adsorption, absorption, distillation, or membrane separation.

7. The microchannel apparatus of claim 1, further comprising a second microchannel reaction section downstream from the microchannel reaction section, wherein the separation section interposes the microchannel reaction section and the second microchannel reaction section.

8. The microchannel apparatus of claim 7, wherein the reaction section includes a catalyst supporting an oxidative coupling of methane reaction.

9. The microchannel apparatus of claim 7, wherein the separation section includes a microchannel.

10. The microchannel apparatus of claim 9, wherein the microchannel mixing section, the microchannel reaction section, and the separation section are integrated within a single device.

11. The microchannel apparatus of claim 6, wherein the microchannel separation section includes two stages of microchannels, with at least one of the two stages of microchannels supporting separation by phases.

12. The microchannel apparatus of claim 6, wherein the separation section is adapted to carry out at least one of phase separation, adsorption, absorption, distillation, or membrane separation.

13. The microchannel apparatus of claim 1, wherein the microchannel mixing section and the microchannel reaction section are synonymous.

14. A microchannel apparatus comprising:
a conduit including provisions for microchannel mixing, microchannel reaction, microchannel heat transfer, and a separation section;
wherein the microchannel mixing includes direct injection inlets;
wherein the microchannel mixing is downstream from the microchannel reaction;
wherein the separation section is downstream from the microchannel reaction.

15. The microchannel apparatus of claim 14, wherein the conduit includes a catalyst supporting an oxidative coupling of methane reaction.

16. The microchannel apparatus of claim 14, wherein the separation section includes a microchannel.

17. The microchannel apparatus of claim 16, wherein the conduit and the separation section are integrated within a single device.

18. The microchannel apparatus of claim 14, wherein the separation section includes two stages of microchannels, with at least one of the two stages of microchannels supporting separation by phases.

19. The microchannel apparatus of claim 14, wherein the separation section is adapted to carry out at least one of phase separation, adsorption, absorption, distillation, or membrane separation.

20. The microchannel apparatus of claim 14, further comprising a second conduit downstream from the first conduit, the second conduit including provisions for microchannel mixing, microchannel reaction, microchannel heat transfer, wherein the separation section interposes the conduit and the second conduit.

21. The microchannel apparatus of claim 20, wherein the conduit includes a catalyst supporting an oxidative coupling of methane reaction.

22. The microchannel apparatus of claim 20, wherein the separation section includes a microchannel.

23. The microchannel apparatus of claim 22, wherein the conduit and the separation section are integrated within a single device.

24. The microchannel apparatus of claim 19, wherein the microchannel separation section includes two stages of microchannels, with at least one of the two stages of microchannels supporting separation by phases.

25. The microchannel apparatus of claim 19, wherein the separation section is adapted to carry out at least one of phase separation, adsorption, absorption, distillation, or membrane separation.

26. The microchannel apparatus of claim 14, wherein the microchannel mixing section and the microchannel reaction section are synonymous.

27. A method of operating a microchannel apparatus comprising:
directing a plurality of first reactants into communication with a first reaction microchannel;
reacting at least a portion of the plurality of first reactants with a plurality of second reactants to form a plurality of first products, a reaction output of the first reaction microchannel comprising first reactants, second reactants, and first products, where the first reactants within the output have a first downstream reaction concentration;
separating at least a portion of the first products from the reaction output within a separation section, the separation being operative to create a separation output comprising first reactants, second reactants, and first products, where the first reactants within the separation output have a separation concentration higher than the first downstream reaction concentration;
reacting at least a portion of the first reactants of the separation output within a second reaction microchannel to form additional first products.

28. The method of claim 27, wherein a temperature difference between the first reaction microchannel and the separation section is greater than 20C.

29. The method of claim 28, wherein the temperature difference between the first reaction microchannel and the separation section is greater than 100C.

30. The method of claim 27, wherein a fluid contact time for at least a portion of the plurality reactants between an end of the first reaction microchannel and a beginning of the separation section is less than 100 milliseconds.

31. The method of claim 30, wherein the fluid contact time for at least the portion of the plurality reactants between the end of the first reaction microchannel and the beginning of the separation section is less than 50 milliseconds.

32. The method of claim 27, wherein the step of separating at least the portion of the first products from the reaction output within the microchannel separation section comprises at least one of phase separation, absorption, adsorption, distillation, and membrane separation.

33. The method of claim 27, wherein the step of separating at least the portion of the first products from the reaction output within the microchannel separation section includes utilization of an ionic liquid.

34. The method of claim 27, wherein the plurality of first reactants comprises a plurality of hydrocarbon molecules.

35. The method of claim 27, wherein the plurality of hydrocarbon molecules comprises a plurality of methane molecules.

36. The method of claim 27, wherein the plurality of second reactants comprises at least one of diatomic oxygen and molecules including oxygen.

37. The method of claim 27, wherein the step of reacting at least the portion of the plurality of first reactants with the plurality of second reactants to form the plurality of first products includes the step of reacting at least the portion of the plurality of first reactants in the presence of a catalyst with the plurality of second reactants to form the plurality of first products.

38. The method of claim 27, wherein the steps are carried out within an integrated device.

39. The method of claim 27, wherein the separation section includes a microchannel.

40. A method of operating a microchannel apparatus comprising:
carrying out an oxidative coupling of methane reaction within a first microchannel reaction stage;
outputting a first reaction stage output stream from the first microchannel reaction stage comprising products and unreacted reactants;
carrying out a first separation within a first separation stage to separate at least a portion of products, formed during the first stage oxidative coupling of methane reaction from the first reaction stage output stream;
carrying out a second stage oxidative coupling of methane reaction within a second microchannel reaction stage; and
outputting a second reaction stage output stream from the second microchannel reaction stage comprising products and unreacted reactants.

41. The method of claim 40, wherein at least one of the first microchannel reaction stage and the second microchannel reaction stage includes a catalyst promoting the oxidative coupling of methane reaction.

42. The method of claim 40, wherein a temperature difference between the first microchannel reaction stage and the first separation stage is greater than 20C.

43. The method of claim 42, wherein the temperature difference between the first microchannel reaction stage and the first separation stage is greater than 100C.

44. The method of claim 40, wherein a fluid contact time for at least a portion of reactants between an end of the first microchannel reaction stage and a beginning of the first separation stage is less than 100 milliseconds.

45. The method of claim 44, wherein the fluid contact time for at least a portion of reactants between the end of the first microchannel reaction stage and the beginning of the first separation stage is less than 50 milliseconds.

46. The method of claim 40, wherein at least one of the first separation stage and the second separation stage includes at least one of phase separation, absorption, adsorption, distillation, and membrane separation.

47. The method of claim 40, wherein the first separation stage includes utilization of an ionic liquid.

48. The method of claim 40, further comprising the step of carrying out a second separation within a second separation stage to separate at least a portion of products from the second stage oxidative coupling of methane reaction from the second reaction stage output stream.

49. A method of operating a microchannel apparatus comprising:
carrying out an oxidative coupling of methane reaction within a first microchannel reaction stage;
outputting a first reaction stage output stream from the first microchannel reaction stage comprising products and unreacted reactants;
carrying out a first separation within a first separation stage to separate at least a portion of products, formed during the first stage oxidative coupling of methane reaction from the first reaction stage output stream; and
recycling at least a portion of unreacted reactants from the first reaction stage output stream to an input stream for the first microchannel reaction stage.

50. The method of claim 49, wherein the first microchannel reaction stage includes a catalyst promoting the oxidative coupling of methane reaction.

51. The method of claim 49, wherein a temperature difference between the first microchannel reaction stage and the first separation stage is greater than 20C.

52. The method of claim 51, wherein the temperature difference between the first microchannel reaction stage and the first separation stage is greater than 100C.

53. The method of claim 49, wherein a fluid contact time for at least a portion of reactants between an end of the first microchannel reaction stage and a beginning of the first separation stage is less than 100 milliseconds.

54. The method of claim 53, wherein the fluid contact time for at least a portion of reactants between the end of the first microchannel reaction stage and the beginning of the first separation stage is less than 50 milliseconds.

55. The method of claim 49, wherein the first separation stage includes at least one of phase separation, absorption, adsorption, distillation, and membrane separation.

56. The method of claim 49, wherein the first separation stage includes utilization of an ionic liquid.

57. The method of claim 27, wherein the first separation stage includes microchannels.

58. The method of claim 49, wherein the first separation stage includes microchannels.

59. The method of claim 14, further comprising the step of cooling products from the microchannel reaction prior to reaching the microchannel separation section.

60. The method of claim 27, further comprising the step of cooling the first reaction stage output stream prior to reaching the first separation stage.

61. The method of claim 49, further comprising the step of cooling the reaction output prior to reaching the first separation stage.

62-91. (canceled)

92. A method of operating a microchannel apparatus comprising:
directing a first reactant stream into communication with a catalyst, the first reactant stream comprising a plurality of first reactants;
reacting at least a portion of the plurality of first reactants with a plurality of second reactants comprising a second reactant stream while in the presence of the catalyst to form a plurality of first products;
reacting at least a portion of the first products from remaining first reactants and second reactants downstream by reacting the at least a portion of the first products with a plurality of third reactants to form a plurality of second products;
separating some of the second products from the first products using a separation medium which performs preferential separation of either the first or second products with respect to the other reactants or products.

93. The method of claim 92, wherein the separation medium is one of a solvent, a membrane, and a sorbent.

94. The method of claim 93, wherein the solvent is introduced downstream of the catalyst and mixed with the plurality of first products.

95. The method of claim 92, wherein the separation medium comprises a phase separation section of the microchannel apparatus.
